(19) United States
(12) Patent Application Publication
(10) Pub. No.: US 2021/0024636 A1
(43) Pub. Date: Jan. 28, 2021

(54) COMPOSITIONS AND METHODS FOR TREATING SEXUAL DYSFUNCTION

(71) Applicant: ChemistryRX, Philadelphia, PA (US)

(72) Inventor: Amany Mansour-Awad, Philadelphia, PA (US)

(21) Appl. No.: 16/939,657

(22) Filed: Jul. 27, 2020

Related U.S. Application Data
(60) Provisional application No. 62/878,828, filed on Jul. 26, 2019.

(51) Int. Cl. C07K 16/28; A61K 9/06; A61K 9/00; A61K 31/4725

(52) U.S. Cl. CPC C07K 16/2842; A61K 9/06; A61K 9/0034; C07K 2317/76; C07K 16/2845; C07K 16/2803; A61K 31/4725

(57) ABSTRACT

Compositions and methods for treating sexual dysfunction using i integrin antagonists, intracellular adhesion molecule antagonists, or leukocyte antagonists are described herein.

### CROSS-REFERENCE TO RELATED APPLICATIONS

This application claims the benefit of U.S. Provisional No. 62/878,828, entitled “Compositions and Methods for Treating sexual Dysfunction,” filed Jul. 26, 2019 the entirety of which is hereby incorporated by reference.

### GOVERNMENT INTERESTS

Not applicable

### PARTIES TO A JOINT RESEARCH AGREEMENT

Not applicable

### INCORPORATION OF MATERIAL ON COMPACT DISC

Not applicable

### BACKGROUND

Sexual dysfunction is a persistent problem that has only recently been addressed with scientific investigation and effective treatment. Male impotence, especially male erectile dysfunction, has received the most attention. Female sexual dysfunction has been considered in the context of male erectile dysfunction, in part because of the anatomical and physiological parallels between the male and female genitalia, and in part, with the hope that effective treatments for male erectile dysfunction could provide some relief for female sexual dysfunction. Yet, female sexual dysfunction manifests in physiologically different ways than male erectile dysfunction. It may be expressed as vaginal dryness, lack of subjective excitement, lack of genital response, such as lubrication and swelling, or lack of other somatic responses.

While increased understanding of the pathophysiology of male erectile dysfunction has progressed rapidly in the past decade and led to new therapeutic modalities, little has been done to address similar issues in women.

### SUMMARY OF THE INVENTION

Various embodiments are directed to compositions for treating sexual dysfunction including an effective amount of an integrin antagonist and a pharmaceutically acceptable excipient, carrier, or diluent. In some embodiments, the integrin antagonists may be natalizumab, vedolizumab, etrolizumab, AMG 181, AJM 300, PF-00547659, enlimomab, lifitegrast, and the like and combinations thereof. In some embodiments, the integrin antagonist may have a concentration of about 1 wt. % to about 25 wt., and in certain embodiments, the effective amount may be about 1 mg to about 1000 mg integrin antagonist.

In various embodiments, the composition may be in the form of creams, lotions, foams, liniments, balms, ointments, gels, and combinations thereof. Such compositions may include a base such as, for example, white petrolatum, white petrolatum USP, mineral jelly, petroleum jelly, yellow petrolatum, yellow soft paraffin, white soft paraffin, fats, waxes, sterols, fat-soluble vitamins, monoglycerides, diglycerides, triglycerides, phospholipids, and combinations thereof, and such compositions may further include pharmaceutically acceptable diluents, fillers, disintegrants, binders, lubricants, surfactants, hydrophobic vehicles, water soluble vehicles, emulsifiers, buffers, humectants, moisturizers, solubilizers, preservatives, colorants, plasticizers, carriers, excipients, and and combinations thereof.

Further embodiments are directed to methods for treating a sexual dysfunction by administering a therapeutically effective amount of a composition comprising integrin antagonists to a subject in need of treatment. In some embodiments, the integrin antagonists may be natalizumab, vedolizumab, etrolizumab, AMG 181, AJM 300, PF-00547659, enlimomab, lifitegrast, and the like and combinations thereof. In some embodiments, the integrin antagonist may have a concentration of about 1 wt. % to about 25 wt., and in certain embodiments, the effective amount may be about 1 mg to about 1000 mg integrin antagonist.

In various embodiments, the composition may be in the form of creams, lotions, foams, liniments, balms, ointments, gels, and combinations thereof. Such compositions may include a base such as, for example, white petrolatum, white petrolatum USP, mineral jelly, petroleum jelly, yellow petrolatum, yellow soft paraffin, white soft paraffin, fats, waxes, sterols, fat-soluble vitamins, monoglycerides, diglycerides, triglycerides, phospholipids, and combinations thereof, and such compositions may further include pharmaceutically acceptable diluents, fillers, disintegrants, binders, lubricants, surfactants, hydrophobic vehicles, water soluble vehicles, emulsifiers, buffers, humectants, moisturizers, solubilizers, preservatives, colorants, plasticizers, carriers, excipients, and and combinations thereof.

In some embodiments, administering may include applying the composition to female genitalia. The subject of various embodiments may experience or be experiencing menopause, stress, medication use, and combinations thereof. The step of administering may be carried out before or during sexual intercourse, and in some embodiments, such methods may include readministering the composition.

### DESCRIPTION OF THE DRAWINGS

Not applicable

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers; reference to an “excipient” includes a single excipient as well as two or more of the same or different excipients, and the like.

The compositions of the present disclosure can comprise, consist essentially of, or consist of, the components disclosed.

All percentages, parts and ratios are based upon the total weight of the topical compositions and all measurements made are at about 25° C., unless otherwise specified.

The word “about” when immediately preceding a numerical value means a range of plus or minus 10% of that value, e.g, “about 50” means 45 to 55, “about 25,000” means 22,500 to 27,500, etc, unless the context of the disclosure indicates otherwise, or is inconsistent with such an interpretation. For example in a list of numerical values such as “about 49, about 50, about 55, “about 50” means a range extending to less than half the interval(s) between the preceding and subsequent values, e.g, more than 49.5 to less than 52.5. Furthermore, the phrases “less than about” a value or “greater than about” a value should be understood in view of the definition of the term “about” provided herein.

The terms “administer,” “administering” or “administration” as used herein refer to either directly administering a compound (also referred to as an agent of interest) or pharmaceutically acceptable salt of the compound (agent of interest) or a composition to a subject.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical, cosmetic or other agent across a tissue layer such as the stratum corneum or stratum spinosum.

The term “disorder” is used in this disclosure to mean, and is used interchangeably with the terms disease, condition, or illness, unless otherwise indicated.

The terms “effective amount” and “therapeutically effective amount” are used interchangeably in this disclosure and refer to an amount of a compound that, when administered to a subject, is capable of reducing a symptom of a disorder in a subject. The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “pharmaceutically acceptable” is employed herein to refer to those agents of interest/compounds, salts, compositions, dosage forms, etc, which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, “pharmaceutically acceptable” means approved by a regulatory agency of the federal or a state government, or listed in the U.S. Pharmacopeia or other generally recognized pharmacopeia for use in mammals (e.g, animals), and more particularly, in humans.

The term “salts” as used herein embraces pharmaceutically acceptable salts commonly used to form alkali metal salts of free acids and to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid. Examples of such inorganic acids are hydrochloric, hydrobromic, hydroiodic, nitric, carbonic, sulfuric, and phosphoric acid. Appropriate organic acids can be selected from aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid.

The term “patient” and “subject” are interchangeable and may be taken to mean any living organism which may be treated with compounds of the present invention. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is a human.

The term “treating” is used herein, for instance, in reference to methods of treating a skin disorder or a systemic condition, and generally includes the administration of a compound or composition which reduces the frequency of, or delays the onset of, symptoms of a medical condition in a subject relative to a subject not receiving the compound or composition. This can include reversing, reducing, or arresting the symptoms, clinical signs, and underlying pathology of a condition in a manner to improve or stabilize a subject's condition.

As used herein, terms such as “genital lubricant,” “vaginal lubricant,” and “topical use” refer to a composition that is administered to and spread across the surface of the skin or a mucous membrane (by contrast, “systemic” administration refers to a drug or other compound that is ingested orally or injected beneath the skin).

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason. Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Various embodiments of the invention are directed to genital lubricants compositions containing integrin antagonists, intracellular adhesion molecules antagonists, or leukocyte antagonists (“integrin antagonists”) and methods for making such compositions. Additional embodiments include methods for treating female sexual dysfunction by administering such compositions. The integrin antagonists may be small molecule drugs, macromolecular drugs, biologics, antibodies, chimeric antibodies, peptides, and the like and combinations thereof.

In some embodiments the method may include administering the composition to the internal or external vaginal tissue prior to or during sexual intercourse. The compositions of the invention may be useful when used as a vaginal moisturizer or personal lubricant for use prior or during sexual intercourse. The composition of the invention may provide safe and immediate relief of sexual dysfunction such as vaginal dryness, as a result of, for example, menopause, stress, medication use and the like and combinations thereof. Such compositions may be helpful in preventing and diminishing the sexually related problems such as loss of libido, frigidity, lack of vaginal lubrication, and impotence and the like and combinations thereof when used on a regular basis.

The integrin antagonists of embodiments are not limited and can be any compound or composition that is capable of binding to integrin and causing a physiological response. Such integrin antagonists may also be referred to as intracellular adhesion molecule antagonists, referring to the function of integrins as intracellular adhesion molecules, or leukocyte antagonists based on the physiological response initiated by these compounds or compositions. For example, in some embodiments, the integrin antagonists may be natalizumab, vedolizumab, etrolizumab, AMG 181, AJM 300, PF-00547659, enlimomab, lifitegrast, and the like and combinations thereof. In certain embodiments, the integrin antagonist may be lifitegrast. Lifitegrast is known to bind to the integrin lymphocyte function-associated antigen-1 (LFA-1), a cell surface protein found on leukocytes and blocks the interaction of LFA-1 with its cognate ligand intercellular adhesion molecule-1 (ICAM-1). LFA-1/ICAM-1 interaction can contribute to the formation of an immunological synapse resulting in T-cell activation and migration to target tissues. In vitro studies demonstrated that lifitegrast may inhibit T-cell adhesion to ICAM-1 in a human T-cell line and may inhibit secretion of inflammatory cytokines in human peripheral blood mononuclear cells. Various embodiments of the invention encompass any compound or composition capable of causing such a response.

The concentration of integrin antagonists in the compositions of the invention may vary among embodiments. For example, in some embodiments, the amount of integrin antagonists in the compositions of embodiments may be about 1 wt. % to about 25 wt. %, about 1 wt. % to about 20 wt. %, about 1 wt. % to about 15 wt. %, about 2 wt. % to about 10 wt. %, about 2 wt. % to about 7.5 wt. %, about 5 wt. %, or any range or individual concentration encompassed by these example ranges. In some embodiments, the integrin antagonists in the compositions of embodiments may have a concentration of about 10 mg/ml to about 200 mg/ml, about 10 mg/ml to about 150 mg/ml, about 10 mg/ml to about 100 mg/ml, about 20 mg/ml to about 75 mg/ml, about 50 mg/ml, or any range or individual concentration encompassed by these example ranges.

The compositions of various embodiments can be in any form, and embodiments include integrin antagonist containing creams, lotions, foams, liniments, balms, ointments, gels, and the like.

Creams refer to semi-solid emulsions of oil and water in approximately equal proportions. They are divided into two types: oil-in-water (O/W) creams, composed of small droplets of oil dispersed in a continuous phase; and water-in-oil (W/O) creams, composed of small droplets of water dispersed in a continuous oily phase. Creams can provide a barrier to protect the skin. This may be a physical barrier or a chemical barrier as with UV-absorbing compounds. To aid in the retention of moisture (especially water-in-oil creams), creams are usually used for a variety of purposes including cleansing, emollient effects, and as a vehicle for drug substances such as local anesthetics, anti-inflammatories (NSAIDs or corticosteroids), hormones, antibiotics, antifungals or counter-irritants.

Lotions are low- to medium-viscosity topical preparation. Most lotions are oil-in-water emulsions containing an emulsifier such as cetyl alcohol to prevent separation of these two phases. Lotions can include fragrances, glycerol, petroleum jelly, dyes, preservatives, proteins and stabilizing agents.

Pharmaceutical foams are pressurized dosage forms containing one or more active ingredients that, upon valve actuation, emit a fine dispersion of liquid and/or solid materials in a gaseous medium. Foam formulations are generally easier to apply, are less dense, and spread more easily than other topical dosage forms. Foams may be formulated in various ways to provide emollient or drying functions to the skin, depending on the formulation constituents. Accordingly, this delivery technology is a useful addition to the spectrum of formulations available for topical use.

Liniments or balms are topical formulations that are of a similar viscosity to lotions and less viscous than an ointment or cream. Liniments are generally applied with friction by rubbing the liniment into the skin. Liniments typically are formulated from alcohol, acetone, or similar quickly evaporating solvents and may contain counterirritant aromatic chemical compounds such as methyl salicilate, benzoin resin, or capsaicin.

Ointments are compositions in which oil and water are provided in a ratio of from 7:1 to 2:1, from 5:1 to 3:1, or 4:1. Ointments are generally formulated using oils, waxes, water, alcohols, petroleum products, water, and other agents to prepare formulations with various viscosities and solvent properties. Commonly used formulations include oleaginous base (White Ointment), absorption base, W/O emulsion base (Cold Cream type base), O/W emulsion base (Hydrophilic Ointment), water soluble base, in addition to others. These preparations are used to dissolve or suspend substances or products with medicinal or cosmetic value.

Example compositions may include various known components. For example, in some embodiments, the composition may include a solvent such as isopropyl alcohol, dipropylene glycol methyl-ether, butylated hydroxytoluene dipropylene glycol monomethyl-ether, 1-methoxy 2-propanol (glysolv PM/lcinol PM), Ethylene glycol monobutylether (butyl glyxolv/butyl icinol), Butyl di glysolv (butyl-icinol), Transcutol, propylene glycol (PG), N-methyl-2 pyrrolidone (NMP), methylene chloride, diethyl ether, ethanol, acetonitrile, ethyl acetate, benzyl alcohol, a combination of natural oil; ethylene glycol, propylene glycol, dimethyl polysiloxane (DMPX), oleic acid, caprylic acid, 1-octanol, ethanol (denatured or anhydrous), liposomal compositions, suitable plant oils, such as Aloe vera derivatives or sesame seed oil or derivatives thereof, acrylic polymers, rubber-based polymers, polysiloxane-based polymers, polyvinylpyrrolidone-based polymers, dimethylsulfoxide (DMSO), dimethylformamide (DMF), dimethylacetamide, N-methyl-2-pyrrolidone, hexamethylphosphoramide (HMPA), lecithin, Transfersomes® (bi-component vesicular aggregates), ethosomes, azone, castor oil derivatives, such as ethoxylated castor oil, jojoba oil derivatives, corn oil derivatives, emu oil derivatives, and the like and combinations thereof. The solvent can be present in a concentration of about 5.0% (w/w) to about 15.0% (w/w)., about 6.0% (w/w) to about 10.0% (w/w), about 7.5% (w/w) to about 10.5% (w/w), about 8.0% (w/w) to about 10.0% (w/w), or any range or individual concentration of solvent encompassed by these example ranges.

In certain embodiments, the compositions may include a base such as, for example, white petrolatum, white petrolatum USP, mineral jelly, petroleum jelly, yellow petrolatum, yellow soft paraffin, white soft paraffin, fats, waxes, sterols, fat-soluble vitamins, monoglycerides, diglycerides, triglycerides, phospholipids, and the like and combinations thereof. In some embodiments, the base may be a liposomal base. Liposomal bases are an emulsion that includes a lipophilic component and an aqueous component that can be in the form of a lotion, a cream, a gel, or a paste. Examples of suitable liposomal bases include PCCA Lipoderm®, Lipoderm ActiveMax™, Anhydrous Lipoderm, and Lipoderm High Molecular Weight™ PCCA. Such liposomal base formulations can include, for example, about 60-80% wt/wt water combined with glycerin, C12-15 alkyl benzoate, glyceryl stearate, dimethicone, cetearyl alcohol, cetearyl glucoside, polyacrylamide, cetyl alcohol, magnesium aluminum silicate, xanthan gum, aloe vera (aloe barbadensis), tocopheryl acetate (vitamin E acetate), prunus amygadalus amara (bitter almond) kernel oil, vitis vinifera (Grape) seed extract, triticum vulgare (wheat) germ oil, retinyl palmitate (vitamin A palmitate), ascorbyl palmitate (vitamin C palmitate), Pro-Lipo Multi-emulsion Liposomic System, tetrasodium EDTA, phenoxyethanol, sodium hydroxymethylglycinate and the like and combinations thereof.

The amount of base in the compositions of embodiments can vary and will depend on the amounts of the other components. More base can be added to compensate for smaller amounts of other components in the desired topical pharmaceutical formulation. In some embodiments, the base may be present in a concentration of about 65% (w/w) to about 90% (w/w) of the total composition, or any range or individual concentration known in the art.

In some embodiments, the compositions may include an antioxidant. Such antioxidant may be, for example, butylated hydroxytoluene, ascorbic acid, ascorbic palmitate, butylated hydroxyanisole, 2,4,5-trihydroxybutyrophenone, 4-hydroxymethyl-2,6-di-tert-butylphenol, erythorbic acid, gum guaiac, propyl gallate, thiodipropionic acid, dilauryl thiodipropionate, tert-butylhydroquinone, tocopherol, and the like and pharmaceutically acceptable salt or ester thereof or combinations thereof. The antioxidant can be presention in a concentration of about 0.01% (w/w) to about 1% (w/w) of the total composition or any individual concentration encompassed by this example range.

In some embodiments, the composition may include an emulsifying agent including, for example, various monoglycerides, diglycerides, triglycerides, and blends thereof at a concentration of about 3% (w/w) to about 10% (w/w) of the total composition.

In some embodiments, the compositions may further include a humectant that provides soothing, smoothing, moisturizing, or protects the skin. The humectant is not limited and can be, for example, calamine, dodecylsulphate, sodium lauryl sulphate (SLS), a polyoxyethylene ester of polysorbitan, such as monooleate, monolaurate, monopalmitate, monostearate esters, esters of sorbitan, the polyoxyethylenes ethers, the sodium dioctylsulphosuccinate (DOSS), lecithin, and sodium docusate. The amount of humectant in such compositions may be about 0.01% (w/w) to 5% (w/w) of the total composition.

In some embodiments, the composition may further include an analgesic agent such as, for example, methyl salicylate, codeine, morphine, methadone, pethidine, buprenorphine, hydromorphine, levorphanol, oxycodone, fentanyl, a non-steroidal anti-inflammatory drug (NSAID), and the like and cobinations thereof. The amount of the analgesic agent such compositions may be about 0.01% (w/w) to 5% (w/w) of the total composition.

In some embodiments, a suitable thickening agent which is widely used in genital lubricants comprises chemically treated derivatives of cellulose (such as hydroxyethyl- or hydroxymethyl-cellulose). Other thickening agents which have been used in skin-contacting compounds, and which offer candidate agents for potential use in genital lubricant compositions, include acacia, agar, alginate, carrageenan, gum tragacanth, xanthan gum, collagen, carboxypolymethylene, glyceryl monostearate, polyvinylpyrrolidone, and polyacrylamide, and the like and combinations thereof.

In certain embodiments, the preferred candidates for use as lubricating agents in the composition of this invention are glycerin, propylene glycol, polyethylene glycol, and polypropylene glycol, as they have long been used in sexual lubricants and other skin-contacting formulations with no adverse effects. The suitability of any other candidate lubricating agent can be determined through routine experimentation in humans to ensure that it will not cause irritation or other adverse effects, and in in vitro cell culture and in in-vivo lab animal tests.

In contrast to non-physiological lubricants such as motor oil, physiologically acceptable lubricating agents should be either gradually broken down into innocuous substances in the body if they are absorbed by tissue to a significant degree through the skin or mucous membranes, or they should be of a nature that allows them to be secreted by the vagina and washed cleanly from the skin, so that they will not foul and clog the pores in membranes or dermal layers. Several lubricating agents which are used in commercially available sexual lubricants satisfy these criteria, including glycerin (also called glycerine, glycerol, 1,2,3-propanetriol, and trihydroxypropane) and certain types of polyethylene glycol (PEG), such as PEG 200 or PEG 400 (the numbers indicate different molecular weight averages). Various other polymers (such as polypropylene glycol, polyisobutene, and polyoxyethylene) and behenic acid and behenyl alcohol are also used as lubricants in cosmetics and other formulations that contact the skin. In addition, some sugar-alcohols such as sorbitol, and some silicon compounds such as polydimethylsiloxane, are also used as skin-contacting lubricating agents

Other components, including preservatives (such as DMDM hydantoin, chlorhexidine gluconate), anti-crystallization agents (such as glucono-delta-lactate), fragrances, sweeteners, odorants, coloring agents, alkaline or acidic or buffering agents to maintain the proper pH (such as EDTA), and soothing, anti-swelling agents (such as lanolin, aloe vera extract, or hydrocortisone), antiviral agents (such as zinc salts; see U.S. Pat. No. 5,785,054), hormones (such as estrogen) or spermicide (such as nonoxynol-9) can be added to the lubricant composition of the invention described herein. However, at the concentrations used, any such additive should not seriously impede the desired activity of the final composition and should not irritate or have other adverse effects on the genitals.

In various embodiments, the composition may be in the form of a lotion, cream, a gel, and the like, and may contain various physiologically acceptable carriers, excipients, diluents, and the like and combinations thereof.

In some embodiments, the compositions described above may be formulated as a liquid. Liquid dosage forms for topical administration may include diluents such as, for example, alcohols, glycols, oils, water, and the like. Such compositions may also include wetting agents or emulsifiers. In some embodiments, the compositions of embodiments may be formulated as oil-in-water or water-in-oil emulsion. A cream can be a water-in-oil (w/o) emulsion in which an aqueous phase is dispersed in an oil phase, or an oil-in-water (o/w) emulsion in which an oil is dispersed within an aqueous base. An ointment generally refers to a more viscous oil-in-water cream. Traditional ointment bases (i.e. carrier) include hydrocarbons (petrolatum, beeswax, etc.) vegetable oils, fatty alcohols (cholesterol, lanoilin, wool alcohol, stearyl alcohol, etc.) or silicones. Insoluble solids such as starch, zinc oxide, calcium carbonate, or talc can also be used in ointments and creams. Gel forms of the compositions described above can be formed by the entrapment of large amounts of aqueous or aqueous-alcoholic liquids in a network of polymers or of colloidal solid particles. Such polymers or colloids (gelling or thickening agents) are typically present at concentrations of less than 10% w/w and include carboxymethyl cellulose, hydroxypropylmethyl cellulose, hydroxyethyl cellulose, methyl cellulose, sodium alginate, alginic acid, pectin, tragacanth, carrageen, agar, clays, aluminum silicate, carbomers, and the like.

In some embodiments, the compositions described above may further include one or more pharmaceutically acceptable diluents, fillers, disintegrants, binders, lubricants, surfactants, hydrophobic vehicles, water soluble vehicles, emulsifiers, buffers, humectants, moisturizers, solubilizers, preservatives, colorants, plastizers, carriers, excipients, and the like and combinations thereof. The person of ordinary skill in the art can refer to various pharmacologic references such as, for example, Modern Pharmaceutics, Banker & Rhodes, Marcel Dekker, Inc. (1979) and Goodman & Gilman's The Pharmaceutical Basis of Therapeutics, 6th Edition, MacMillan Publishing Co, New York (1980) for guidance in determining the amount of such components in the compositions and formulations of embodiments.

Emollient or lubricating vehicles that help hydrate the skin can also be used. Examples of suitable bases or vehicles for preparing hydrating compositions for use with human skin are petrolatum, petrolatum plus volatile silicones, lanolin, cold cream (USP), and hydrophilic ointment (USP).

Vitamins include, for example, vitamin D, vitamin K, vitamin B (including niacinamide, nicotinic acid, C₁₋₁₈ nicotinic acid esters, and nicotinyl alcohol; B6 compounds, such as pyroxidine; and B5 compounds, such as panthenol, or “pro-B5”), vitamin A (including retinoids such as retinyl propionate, carotenoids, and other compounds), vitamin E (including tocopherol sorbate, tocopherol acetate, other esters of tocopherol), vitamin C (including ascorbyl esters of fatty acids, and ascorbic acid derivatives, for example, ascorbyl glucoside, magnesium ascorbyl phosphate, sodium ascorbyl phosphate, and ascorbyl sorbate), and all natural and/or synthetic analogs thereof, and combinations thereof. In various embodiments, the compositions may include about 0.0001 wt. % to about 50 wt. %, about 0.001 wt. % to about 10 wt. %, about 0.01 wt. % to about 5 wt. %, or about 0.1 wt. % to about 1 wt. %, or any individual concentration or range of each vitamin contained in the composition.

Peptides include di-, tri-, tetra-, penta-, and hexa-peptides, their salts, isomers, derivatives, and mixtures thereof. Examples of useful peptide derivatives include, but are not limited to, peptides derived from soy proteins, palmitoyl-lysine-threonine (pal-KT) and palmitoyl-lysine-threonine-threonine-lysine-serine (MATRIXYL®) palmitoyl-glycine-glutamine-proline-arginine (RIGIN®), these three being available from Sederma, France, and Cu-histidine-glycine-glycine (Cu-HGG, also known as IAMIN®), and naturally occurring and synthesized derivatives thereof, and combinations thereof. In various embodiments, the compositions may include about 1×10⁻⁷ wt. % to about 20 wt. %, about 1×10⁻⁶ wt. % to about 10 wt. %, and about 1×10⁻⁵ wt. % to about 5 wt. %, or any individual concentration or range of each peptide contained in the composition.

The compositions of embodiments described above may enhance the strength of known topical active agent thereby reducing the necessary dosage required to achieve a therapeutically effective amount. For example, in some embodiments, the strength of a composition containing an active agent may be about equal to about 80% or 90% greater than the active agent delivered in a standard topical formulation. In other embodiments, the strength of a composition containing an active agent may be about equal to about 75% greater, about 1.0% to about 80% greater, about 1.0% to about 75% greater, about 1.0% to about 50% greater, about 1.0% to about 25% greater, about 2.0% to about 80% greater, about 2.0% to about 75% greater, about 2.0% to about 50% greater, about 2.0% to about 25% greater, about 5.0% to about 50% greater, about 5.0% to about 25% greater, or any range or individual strength encompassed by these example ranges. Thus, the compositions described herein may provide therapeutic equivalence of known topically administered active agents with that an administered dose that is equal to or at least about 75% less than a standard dose, equal to or about 50% less than a standard dose, equal to or about 25% less than a standard dose, equal to or about 10% less than a standard dose, about 1.0% to about 75% less than a standard dose, about 1.0% to about 50% less than a standard dose, about 1.0% to about 25% less than a standard dose, about 1.0% to about 10% less than a standard dose, about 2.0% to about 75% less than a standard dose, about 2.0% to about 50% less than a standard dose, about 2.0% to about 25% less than a standard dose, about 2.0% to about 10% less than a standard dose, or any range or individual value encompassed by these example ranges.

A wide variety of methods may be used for preparing the formulations described above. Broadly speaking, the formulations may be prepared by combining together the components of the formulation, as described herein, at a temperature and for a time sufficient to provide a pharmaceutically acceptable composition. For example, in some embodiments, the compositions components of the compositions may be dissolved, suspended, dispersed or otherwise mixed in a selected carrier or vehicle, at an effective concentration such that the condition to be treated is relieved or ameliorated.

Additional embodiments include methods for treating female sexual dysfunction. Such methods may include the steps of administering a topical composition containing an effective amount of integrin antagonists to female genitalia. Administrating the composition can be carried out on internal vaginal tissue, external vaginal tissue, or combinations thereof. In various embodiments, the step of administering can be carried out by applying the composition to female genitalia by rubbing it onto the female genitalia, spraying it onto the female genitalia, or otherwise contacting the female genitalia with the composition. The subject of such treatment may exhibit various symptoms associated with female sexual dysfunction including, for example, vaginal dryness, lack of subjective excitement, lack of genital response, such as lubrication and swelling, lack of other somatic responses, and the like and combinations thereof. Sexual dysfunction may occur for any reason including, for example, menopause, stress, medication use, and the like and combinations thereof.

The integrin antagonist may be any of the compounds or combinations of compounds described above in the concentrations described above. The composition may be formulated as creams, lotions, foams, liniments, balms, ointments, gels, and the like containing any of the various components as discussed above. In some embodiments, the composition may be applied to female genitalia or in anticipation of intercourse, before intercourse, or during intercouse, and in some embodiments, the composition may be applied 1, 2, 3, 4, 5, 6, 7, or more times as needed. In certain embodiments, the compositions may be applied 1, 2, 3, 4, 5, 6, 7, or more times per week.

1. A composition for treating sexual dysfunction comprising an effective amount of an integrin antagonist and a pharmaceutically acceptable excipient, carrier, or diluent.

2. The composition of claim 1, wherein the integrin antagonists is selected from the group consisting of natalizumab, vedolizumab, etrolizumab, AMG 181, AJM 300, PF-00547659, enlimomab, lifitegrast, and combinations thereof.

3. The composition of claim 1, wherein the integrin antagonist has a concentration of about 1 wt. % to about 25 wt. %.

4. The composition of claim 1, wherein the effective amount comprises about 1 mg to about 1000 mg integrin antagonist.

5. The composition of claim 1, wherein the composition is in the form of creams, lotions, foams, liniments, balms, ointments, gels, and combinations thereof.

6. The composition of claim 1, further comprising a base selected from the group consisting of white petrolatum, white petrolatum USP, mineral jelly, petroleum jelly, yellow petrolatum, yellow soft paraffin, white soft paraffin, fats, waxes, sterols, fat-soluble vitamins, monoglycerides, diglycerides, triglycerides, phospholipids, and combinations thereof.

7. The composition of claim 1, further comprising pharmaceutically acceptable diluents, fillers, disintegrants, binders, lubricants, surfactants, hydrophobic vehicles, water soluble vehicles, emulsifiers, buffers, humectants, moisturizers, solubilizers, preservatives, colorants, plasticizers, carriers, excipients, and and combinations thereof.

8. A method for treating a sexual dysfunction comprising administering a therapeutically effective amount of a composition comprising integrin antagonists to a subject in need of treatment.

9. The method of claim 8, wherein the integrin antagonists are selected from the group consisting of natalizumab, vedolizumab, etrolizumab, AMG 181, AJM 300, PF-00547659, enlimomab, lifitegrast, and combinations thereof.

10. The method of claim 8, wherein the integrin antagonist has a concentration of about 1 wt. % to about 25 wt. %.

11. The method of claim 8, wherein the effective amount comprises about 1 mg to about 1000 mg integrin antagonist.

12. The method of claim 8, wherein the composition is in the form of creams, lotions, foams, liniments, balms, ointments, gels, and combinations thereof.

13. The method of claim 8, wherein the composition further comprising a base selected from the group consisting of white petrolatum, white petrolatum USP, mineral jelly, petroleum jelly, yellow petrolatum, yellow soft paraffin, white soft paraffin, fats, waxes, sterols, fat-soluble vitamins, monoglycerides, diglycerides, triglycerides, phospholipids, and combinations thereof.

14. The method of claim 8, wherein the composition further comprising pharmaceutically acceptable diluents, fillers, disintegrants, binders, lubricants, surfactants, hydrophobic vehicles, water soluble vehicles, emulsifiers, buffers, humectants, moisturizers, solubilizers, preservatives, colorants, plasticizers, carriers, excipients, and and combinations thereof.

15. The method of claim 8, wherein administering comprises applying the composition to female genitalia.

16. The method of claim 8, wherein the subject experiences menopause, stress, medication use, and combinations thereof.

17. The method of claim 8, wherein administrating is carried out before or during sexual intercourse.

18. The method of claim 8, further comprising readministering the composition.
